(19) United States
(12) Patent Application Publication
(10) Pub. No.: US 2026/0060806 A1
(43) Pub. Date: Mar. 5, 2026

(54) METHOD AND SYSTEM FOR CERVICAL BLADED IMPLANT

(71) Applicant: Evolution Spine, LLC, Dallas, TX (US)

(72) Inventors: Francia Sanchez Villasana, Knoxville, TN (US); James Mirda, Germantown, TN (US); Benjamin Verhoogh Keller, Raleigh, NC (US)

(21) Appl. No.: 19/313,990

(22) Filed: Aug. 29, 2025

Related U.S. Application Data
(60) Provisional application No. 63/688,352, filed on Aug. 29, 2024.

(51) Int. Cl. A61F 2/30; A61B 17/56; A61B 17/84; A61F 2/44

(52) U.S. Cl. CPC A61F 2/30749; A61B 17/846; A61F 2/447; A61B 2017/564; A61F 2002/30131; A61F 2002/30593; A61F 2002/30879; A61F 2002/30892

(57) ABSTRACT

Provided herein are an anchor and interbody for interbody fusion, including an interbody for insertion between human vertebrae, including an interbody cage including a plurality of anchor receptacles, each anchor receptacle configured to receive an anchor to secure the interbody to a vertebra; and the anchor, including a head configured to engage an anchor receptacle; a curved partial-tubular shank with a convex curve, connected to or integral with the head at a distal end of the head, and extending from the head; a tapered tip portion extending from a distal end of the curved partial-tubular shank; one or more rails integral with or connected to an outside surface of the anchor, extending from a proximal end of head and along the curved partial-tubular shank to a proximal end of the tapered tip portion; and a feature formed from two flat surfaces on an outside of the anchor and a rail.

### CROSS-REFERENCE TO RELATED APPLICATIONS

This application claims priority to U.S. Provisional Application Ser. No. 63/688,352, filed Aug. 29, 2024, the entire contents of which are incorporated herein by reference.

### STATEMENT OF FEDERALLY FUNDED RESEARCH

Not applicable.

### TECHNICAL FIELD OF THE INVENTION

The present invention relates in general to surgery on the human spine, and more particularly, to an anchor for use with an interbody in interbody fusion.

### BACKGROUND OF THE INVENTION

Without limiting the scope of the invention, its background is described in connection with an anchor for use with a cervical interbody as an example.

An intervertebral disc in a human spine may be replaced by a spinal implant to promote fusion of the vertebrae adjacent to the disc.

U.S. Pat. No. 9,877,842, issued to Chataigner et al., is said to disclose anchoring devices for spinal implants, implants, surgical instruments, and surgical systems and methods. In some embodiments, an anchor comprises a stiff plate with a longitudinal axis, configured for penetration of its anterior end into a vertebral surface while its posterior end remains engaged with the implant. An implant may include a locking mechanism for the anchor. An anchor may include an abutment configured to abut a complementary abutment of an implant. In some configurations, inserting an anchor in a passage of an implant may displace a locking mechanism, which may resile and lock the anchor in the implant with complementary abutments of the anchor and implant abutting.

U.S. Pat. No. 10,524,929, issued to Shoshtaev, is said to disclose ALIF spine implants, ALIF installation instruments/tools, and ALIF procedures using the present ALIF implants and present ALIF installation instruments for an anterior lumbar interbody fusion (ALIF) surgical procedure. The ALIF implants are characterized by an ALIF cage and anchoring members. The ALIF installation instruments are characterized by a shaft having an inserter on one end that receives and holds an ALIF cage and anchoring members. The installation instrument allows insertion of the ALIF cage into a vertebral space, the anchoring members to be received in the ALIF cage, and then into vertebral bone. The ALIF cage is preferably, but not necessarily, 3-D printed having a central cavity, an end configured to accept a plurality of anchoring members and direct a portion of the anchoring members up and out of the cavity, a cutout configured to receive an anchoring member retention component, and an anchoring member retention component.

U.S. Pat. No. 11,654,030, issued to Valkoun et al., is said to disclose a standalone anterior lumbar interbody spacer that includes a lumbar spacer body and lumbar plate with a unique locking system to rigidly couple the spacer body and plate via a spring loaded lock tab.

Despite these advances, a need remains for anchors with one or more features that guide the anchors into the interbodies for which they are configured to be used and to prevent rotation or twisting of the anchors during deployment in their respective anchor receptacles within an interbody.

### SUMMARY OF THE INVENTION

As embodied and broadly described herein, an aspect of the present disclosure relates to an anchor and interbody for interbody fusion, comprising an interbody for insertion between vertebrae of a human spine, comprising an interbody cage comprising a plurality of anchor receptacles, each anchor receptacle configured to receive an anchor to secure the interbody to a vertebra; and the anchor, comprising a head configured to engage one of the plurality of anchor receptacles; a curved partial-tubular shank with a convex curve, connected to or integral with the head at a distal end of the head, and extending from the head; a tapered tip portion extending from a distal end of the curved partial-tubular shank; one or more rails integral with or connected to an outside surface of the anchor, extending from a proximal end of head and along the curved partial-tubular shank to a proximal end of the tapered tip portion; and a feature formed from two flat surfaces on an outside of the anchor and a rail at an apex where the two surfaces meet. In one aspect, each of the rails is a cornered rail or a smooth rail. In another aspect, when there are two or more rails, the rails can be separated around the outside surface by an angle of up to 180°. In another aspect, the anchor further comprises a plurality of teeth disposed on an edge of the curved partial-tubular shank. In another aspect, the head comprises a threaded opening. In another aspect, the interbody, the anchor, or both can be made of biocompatible materials, such as, stainless steel, titanium alloys, aluminum alloys, chromium alloys, metal alloys, CoCrMo, hydroxyapetite, polyether ether ketone (PEEK), polyether ketone ketone (PEKK), carbon fiber, ABS plastic, polyurethane, polyethylene, photo-polymer, resin, fiber-encased resinous material, a polymer, natural materials, other biocompatible materials, and combinations thereof. In another aspect, anchor, the interbody, or both can be produced at least in part by traditional subtractive manufacturing, rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.

As embodied and broadly described herein, an aspect of the present disclosure relates to an anchor and interbody kit comprising an interbody for insertion between vertebrae of a human spine, comprising an interbody cage comprising a plurality of anchor receptacles, each anchor receptacle configured to receive an anchor to secure the interbody to a vertebra; the anchor, comprising a head configured to engage one of the plurality of anchor receptacles; a curved partial-tubular shank with a convex curve, connected to or integral with the head at a distal end of the head, and extending from the head; a tapered tip portion extending from a distal end of the curved partial-tubular shank; one or more rails integral with or connected to an outside surface of the anchor, extending from a proximal end of head and along the curved partial-tubular shank to a proximal end of the tapered tip portion and a feature formed from two flat surfaces on an outside of the anchor and a rail at an apex where the two surfaces meet; and one or more tools for manipulating the anchor, the interbody, or both. In one aspect, each of the rails is a cornered rail or a smooth rail. In another aspect, when there are two or more rails, the rails can be separated around the outside surface by an angle of up to 180°. In another aspect the anchor further comprises a plurality of teeth disposed on an edge of the curved partial-tubular shank. In another aspect, the head comprises a threaded opening. In another aspect, the interbody, the anchor, or both can be made of biocompatible materials, such as, stainless steel, titanium alloys, aluminum alloys, chromium alloys, metal alloys, CoCrMo, hydroxyapetite, polyether ether ketone (PEEK), polyether ketone ketone (PEKK), carbon fiber, ABS plastic, polyurethane, polyethylene, photo-polymer, resin, fiber-encased resinous material, a polymer, natural materials, other biocompatible materials, and combinations thereof. In another aspect, the anchor, the interbody, or both can be produced at least in part by traditional subtractive manufacturing, rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.

As embodied and broadly described herein, an aspect of the present disclosure relates to a method for using an anchor and interbody for interbody fusion, comprising providing a patient in need of an interbody fusion; providing an interbody for insertion between vertebrae of a human spine, comprising an interbody cage comprising a plurality of anchor receptacles, each anchor receptacle configured to receive an anchor to secure the interbody to a vertebra; providing the anchor, comprising a head configured to engage one of the plurality of anchor receptacles; a curved partial-tubular shank with a convex curve, connected to or integral with the head at a distal end of the head, and extending from the head; a tapered tip portion extending from a distal end of the curved partial-tubular shank; one or more rails integral with or connected to an outside surface of the anchor, extending from a proximal end of head and along the curved partial-tubular shank to a proximal end of the tapered tip portion; a feature formed from two flat surfaces on an outside of the anchor and a rail at an apex where the two surfaces meet; inserting the interbody between two vertebrae of the patient; and inserting the anchor into one of the plurality of anchor receptacles to secure the interbody to a vertebra. In one aspect, each of the rails is a cornered rail or a smooth rail. In another aspect, when there are two or more rails, the rails can be separated around the outside surface by an angle of up to 180°. In another aspect, the anchor further comprises a plurality of teeth disposed on an edge of the curved partial-tubular shank. In another aspect, the head comprises a threaded opening. In another aspect, the interbody, the anchor, or both can be made of biocompatible materials, such as, stainless steel, titanium alloys, aluminum alloys, chromium alloys, metal alloys, CoCrMo, hydroxyapetite, polyether ether ketone (PEEK), polyether ketone (PEKK), carbon fiber, ABS plastic, polyurethane, polyethylene, photo-polymer, resin, fiber-encased resinous material, a polymer, natural materials, other biocompatible materials, and combinations thereof. In another aspect, the anchor, the interbody, or both can be produced at least in part by traditional subtractive manufacturing, rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.

### BRIEF DESCRIPTION OF THE DRAWINGS

For a more complete understanding of the features and advantages of the present invention, reference is now made to the detailed description of the invention along with the accompanying figures, in which:

FIGS. 1A-1C show an end view, a side view, and a perspective view of an embodiment of the invention.

FIGS. 2A-2C show an end view, a side view, and a perspective view of another embodiment of the invention.

FIGS. 3A-3C show an end view, a side view, and a perspective view of another embodiment of the invention.

FIGS. 4A-4C show an end view, a side view, and a perspective view of another embodiment of the invention.

FIGS. 5A-5C show an end view, a side view, and a perspective view of another embodiment of the invention.

FIGS. 6A-6C show an end view, a side view, and a perspective view of another embodiment of the invention.

FIGS. 7A-7C show an end view, a side view, and a perspective view of another embodiment of the invention.

FIGS. 8A and 8B show an anchor and an interbody of the present invention.

FIG. 9 shows a flowchart for a method embodiment of the present invention.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

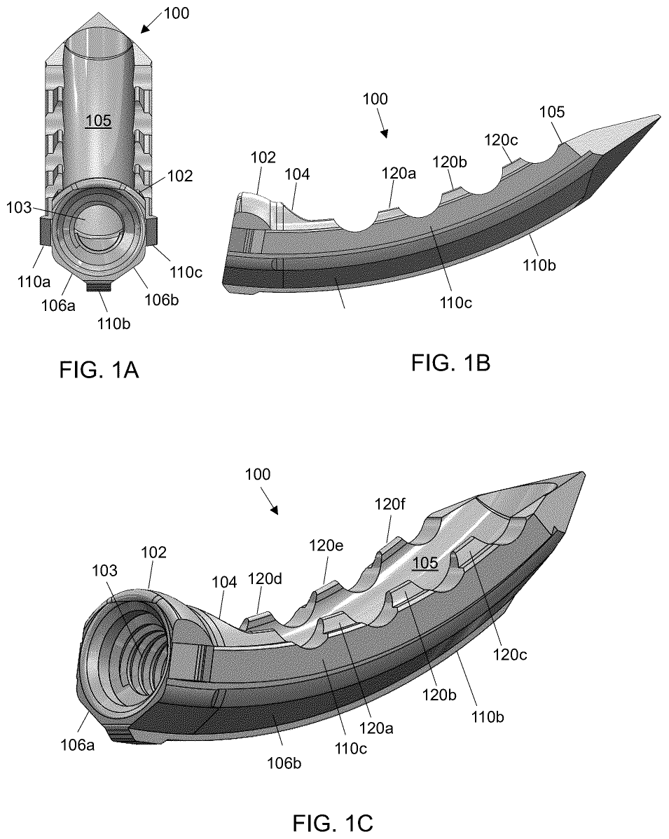

In general terms, embodiments of the present invention include a plurality of anchors (e, g., anchors 100, 200, 300, 400, 500, 600, 700) that are configured to anchor cervical interbodies, which are configured to be implanted between cervical vertebrae to fuse the vertebrae; and a cervical interbody with one or more through anchor receptacles that are configured to receive the plurality of anchors to secure the interbody to the vertebra. Embodiments of the present invention are described herein with reference to cervical interbodies, but the embodiments can include anchors and interbodies for other types of vertebrae and are not limited to use with cervical interbodies and vertebrae. The anchors shown in FIGS. 1A-7C and referenced herein are non-limiting examples of anchors of the present invention.

In some aspects that are shown in FIGS. 1A-7C, an anchor includes a head, a shank, and a tapered tip portion. The head includes a threaded opening that allows for attachment of a removal instrument, a protrusion that serves as a stop, or both. The shank is integral with or connected to a distal end of the head and extending from the head, and the shank has a tapered distal end extending from a distal end of the shank. The shank is partially tubular and curved in a convex manner such that the elongated cavity formed by the partial tube of the shank opens to the inside of the curve of the shank.

In some aspects that are shown in FIGS. 1A-6C, an anchor includes rails as described herein that serve to guide each anchor through the through openings of a cervical interbody with which the anchor is configured to be used and to allow for the anchor to be deployed without twisting or rotating during deployment into the anchor receptacles. The rails also serve to stabilize each anchor in the bone into which it is implanted. Each rail is integral with or connected to an outside surface of the anchor, extending from a proximal end of head and along the curved partial-tubular shank to a proximal end of the tapered tip portion. The anchor can include one or both of two types of rails. The first type of rail has a cross section orthogonal to its longitudinal axis, wherein the cross-section includes part of a polygon and has corners; these are called “cornered rails” herein (e.g., cornered rails 110a, 110b, 110c and other cornered rails described herein). The second type of rail has a cross-section orthogonal to its longitudinal axis, wherein the cross-section is smooth; these are called “smooth rails” herein (e.g., smooth rail 215 and other smooth rails described herein). An anchor can include one or more cornered rails without any smooth rails; one or more smooth rails without any cornered rails; or one or more cornered rails and one or more smooth rails together. Any two rails of either type can be separated by angles around the outer surface such as 5°, 10°, 20°, 30°, 40°, 45°, 50°, 60°, 70°, 80°, 90°, 100°, 110°, 120°, 130°, 135°, 140°, 150°, 160°, 170°, or 180° apart, or generally at spacings up to 180°.

In some aspects, anchors include a “V”-shaped feature to allow an anchor to bank against, ride, run against, slide along, or be supported by its respective anchor receptacle. In these aspects, the “V” feature is formed from two surfaces, which are flat in a cross section orthogonal to a longitudinal axis of the anchor, on the outside of the anchor, and a rail at the apex at which the two flat surfaces meet (e.g., flat surfaces 106a, 106b, and cornered rail 110b). The “V” feature also allows for the anchor to be deployed without twisting or rotating in the anchor receptacles and keeps the anchor centered in the anchor receptacle.

In some aspects, anchors include teeth as described herein (e.g., teeth 120a, 120b, 120c and other teeth described herein). Such teeth aid the anchor in penetrating bone and in holding the anchor in place within the bone. Such teeth can have serrated edges, ridges, or both. The teeth can have various shapes, e.g., triangular, rectangular, polygonal, curved, or some combination of those shapes. An anchor can include one or more teeth disposed on each side of the anchor.

FIGS. 1A-1C show an end view, a side view, and a perspective view of anchor of an embodiment of the invention, the anchor 100, respectively. FIG. 1A shows the anchor 100 from a proximal end of the anchor 100, including the head 102, the threaded opening 103, the shank 105, the flat surfaces 106a, 106b, and the cornered rails 110a, 110b, and 110c. FIG. 1B shows the anchor 100, including the head 102, the protrusion 104, the shank 105, the cornered rails 110b and 110c, and the teeth 120a, 120b, 120c. FIG. 1C shows the anchor 100, including the head 102, the threaded opening 103, the protrusion 104, the shank 105, the flat surfaces 106a, 106b, the cornered rails 110b, 110c, and the teeth 120a, 120b, 120c, 120d, 120e, 120f.

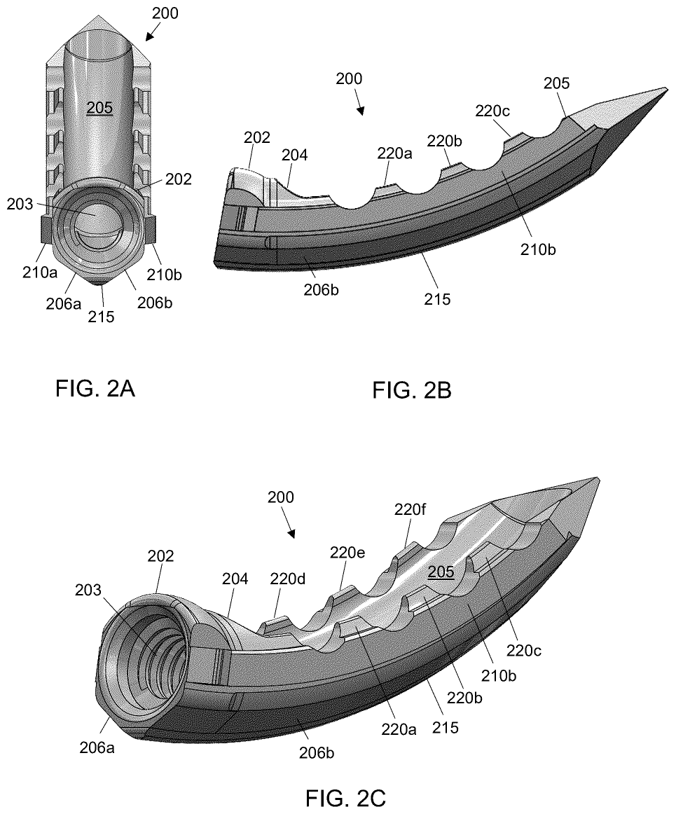

FIGS. 2A-2C show an end view, a side view, and a perspective view of an anchor of another embodiment of the invention, the anchor 200, respectively. FIG. 2A shows the anchor 200, including the head 202, the threaded opening 203, the shank 205, the flat surfaces 206a, 206b, the cornered rails 210a, 210b, and the smooth rail 215. FIG. 2B shows the anchor 200, including the head 202, the protrusion 204, the shank 205, the flat surface 206b, the cornered rail 210b, the smooth rail 215, and the teeth 220a, 220b, 220c. FIG. 2C shows the anchor 200, including the head 202, the threaded opening 203, the protrusion 204, the shank 205, the flat surfaces 206a, 206b, the cornered rail 210b, the smooth rail 215, and the teeth 220a, 220b, 220c, 220d, 220e, 220f.

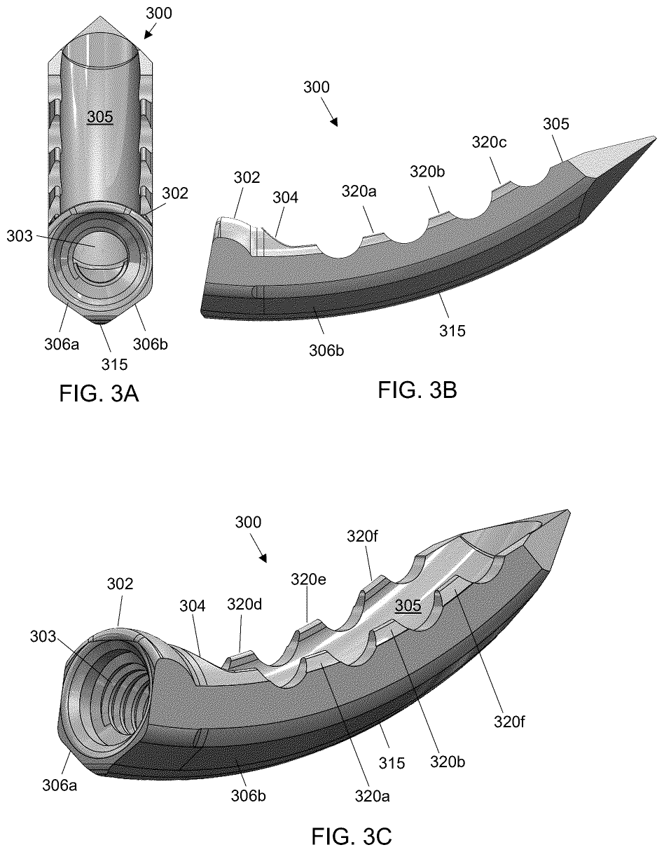

FIGS. 3A-3C show an end view, a side view, and a perspective view of another embodiment of the invention, the anchor 300, respectively. FIG. 3A shows the anchor 300, including the head 302, the threaded opening 303, the shank 305, the flat surfaces 306a, 306b, and the smooth rail 315. FIG. 3B shows the anchor 300, including the head 302, the protrusion 304, the shank 305, the flat surface 306b, the smooth rail 315, and the teeth 320a, 320b, 320c. FIG. 3C shows the anchor 300, including the head 302, the threaded opening 303, the protrusion 304, the shank 305, the flat surface 306b, the smooth rail 315, and the teeth 320a, 320b, 320c, 320d, 320e, 320f.

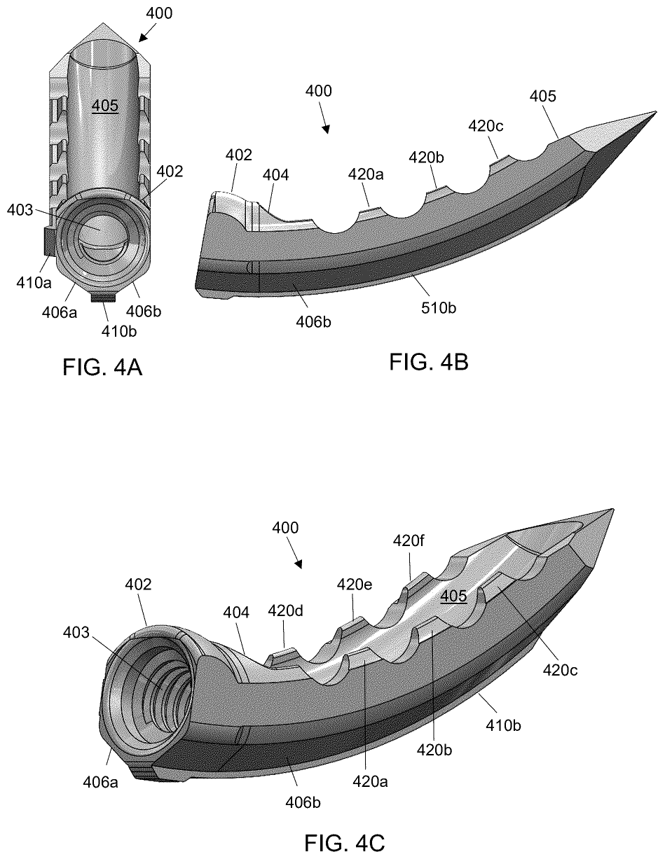

FIGS. 4A-4C show an end view, a side view, and a perspective view of another embodiment of the invention, the anchor 400, respectively. FIG. 4A shows the anchor 400, including the head 402, the threaded opening 403, the shank 405, the flat surface 406a, 406b, and the cornered rails 410a, 410b. FIG. 4B shows the anchor 400, including the head 402, the protrusion 404, the shank 405, the flat surface 406b, the cornered rail 510b, and the teeth 420a, 420b, 420c. FIG. 4C shows the anchor 400, including the head 402, the threaded opening 403, the protrusion 404, the shank 405, the flat surfaces 406a, 406b, the cornered rail 410b, and the teeth 420a, 420b, 420c, 420d, 420e, 420f.

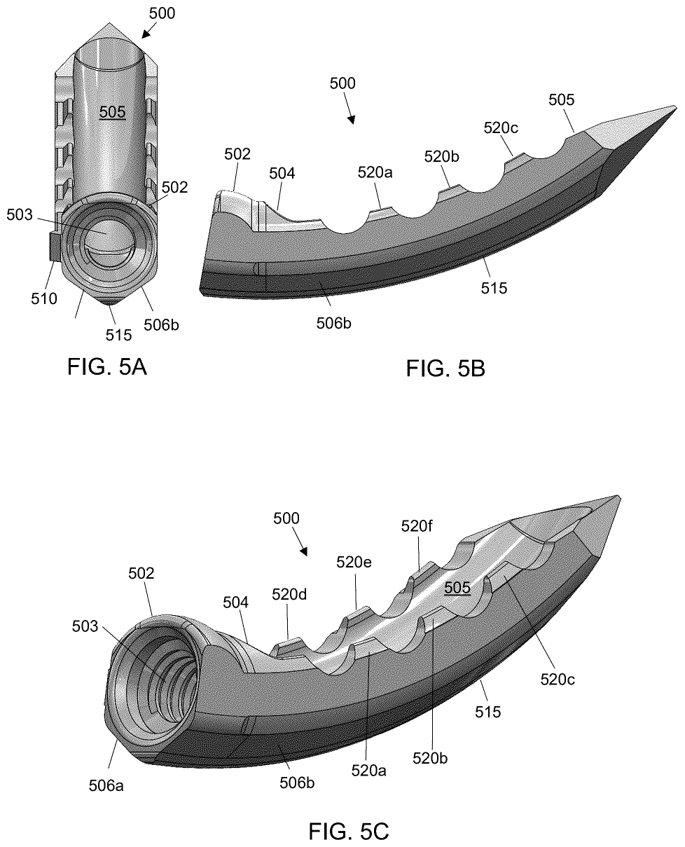

FIGS. 5A-5C show an end view, a side view, and a perspective view of another embodiment of the invention, the anchor 500, respectively. FIG. 5A shows the anchor 500, including the head 502, the threaded opening 503, the shank 505, the flat surfaces 506a, 506b, the cornered rail 510, and the smooth rail 515. FIG. 5B shows the anchor 500, including the head 502, the protrusion 504, the shank 505, the flat surface 506b, the smooth rail 515, and the teeth 520a, 520b, 520c. FIG. 5C shows the anchor 500, including the head 502, the threaded opening 503, the protrusion 504, the shank 505, the flat surfaces 506a, 506b, the smooth rail 515, and the teeth 520a, 520b, 520c, 520d, 520e, 520f.

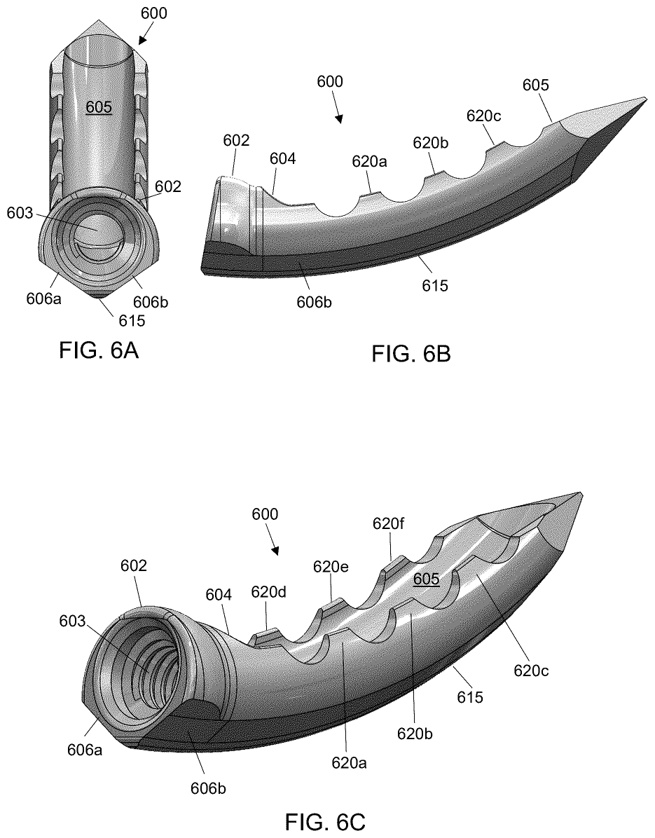

FIGS. 6A-6C show an end view, a side view, and a perspective view of another embodiment of the invention, the anchor 600, respectively. FIG. 6A shows the anchor 600, including the head 602, the threaded opening 603, the shank 605, the flat surfaces 606a, 606b, and the smooth rail 615. FIG. 6B shows the anchor 600, including the head 602, the protrusion 604, the shank 605, the flat surface 606b, the smooth rail 615, and the teeth 620a, 620b, 620c. FIG. 6C shows the anchor 600, including the head 602, the threaded opening 603, the protrusion 604, the shank 605, the flat surfaces 606a, 606b, the smooth rail 615, and the teeth 620a, 620b, 620c, 620d, 620e, 620f.

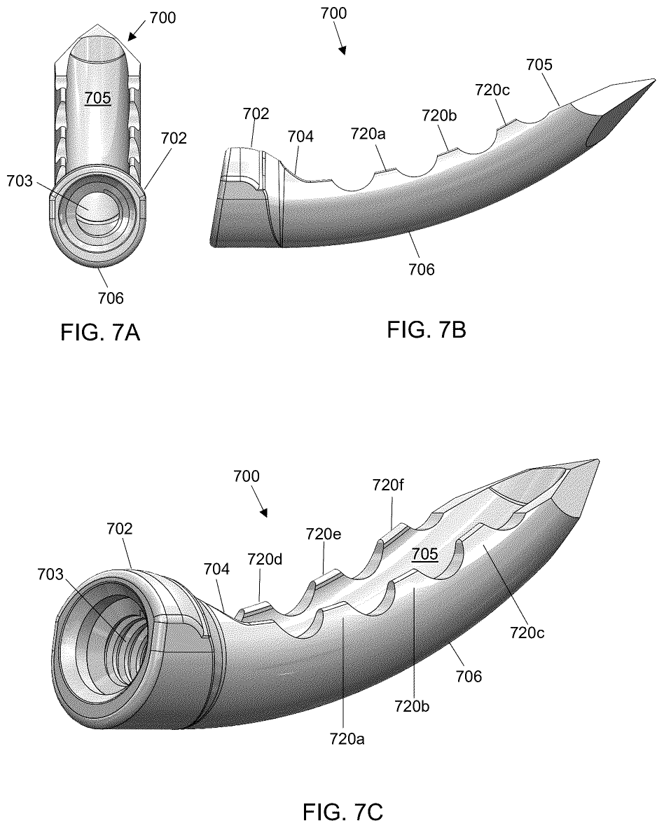

FIGS. 7A-7C show an end view, a side view, and a perspective view of another embodiment of the invention, the anchor 700, respectively. FIG. 7A shows the anchor 700, including the head 702, the threaded opening 703, the shank 705, and an outside surface 706 of the anchor, without rails. FIG. 7B shows the anchor 700, including the head 702, the protrusion 704, the shank 705, the outside surface 706, and the teeth 720a, 720b, and 720c. FIG. 7C shows the anchor 700, including the head 702, the threaded opening 703, the protrusion 704, the shank 705, the outside surface 706, and the teeth 720a, 720b, 720c, 720d, 720e, 720f.

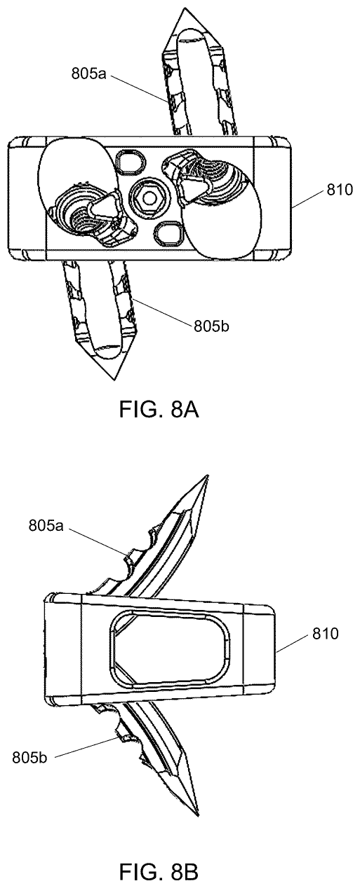

FIGS. 8A and 8B show anchors and an interbody of the present invention. Anchors 805a, 805b are shown inserted into the interbody 810 as they are disposed after an implantation of the interbody 810 and the anchors 805a, 805b is complete.

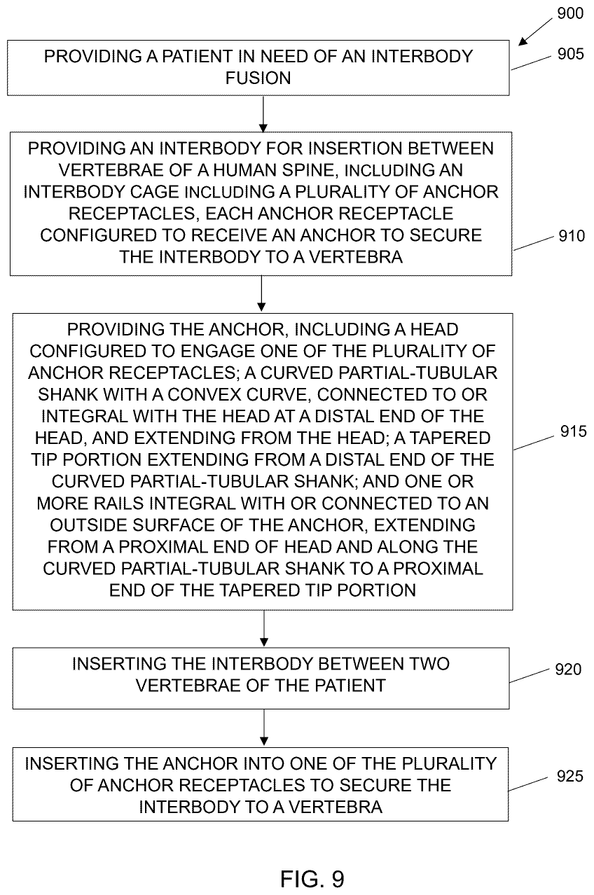

FIG. 9 shows a flowchart for a method embodiment of the present invention. Method 900 includes block 905, which includes providing a patient in need of an interbody fusion. Block 910 includes providing an interbody for insertion between vertebrae of a human spine, including an interbody cage including a plurality of anchor receptacles, each anchor receptacle configured to receive an anchor to secure the interbody to a vertebra. Block 915 includes providing the anchor, including a head configured to engage one of the plurality of anchor receptacles; a curved partial-tubular shank with a convex curve, connected to or integral with the head at a distal end of the head, and extending from the head; a tapered tip portion extending from a distal end of the curved partial-tubular shank; and one or more rails integral with or connected to an outside surface of the anchor, extending from a proximal end of head and along the curved partial-tubular shank to a proximal end of the tapered tip portion. Block 920 includes inserting the interbody between two vertebrae of the patient; and Block 925 includes inserting the anchor into one of the plurality of anchor receptacles to secure the interbody to a vertebra.

The anchors and interbodies described herein (e.g., anchor 100) can be made of biocompatible materials, such as, stainless steel, titanium alloys, aluminum alloys, chromium alloys, metal alloys, CoCrMo, hydroxyapetite, polyether ether ketone (PEEK), polyether ketone ketone (PEKK), carbon fiber, ABS plastic, polyurethane, polyethylene, photo-polymer, resin, fiber-encased resinous material, a polymer, natural materials, other biocompatible materials, and combinations thereof. The anchors and interbodies can be produced at least in part by traditional subtractive manufacturing, rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.

Some embodiments of the present invention include an kit including an anchor (e.g., anchors 100, 200, 300, 400, 500, 600, or 700), an interbody, or both, and one or more tools to manipulate the anchor, one or more tools to manipulate the interbody, or both.

While various features of the present invention are discussed and illustrated separately for clarity, each and every feature disclosed herein can be combined with any other disclosed feature in an embodiment unless such a combination in physically impossible.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property(ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112(f) as it exists on the date of filing hereof unless the words “means for” or “step for” are explicitly used in the particular claim.

What is claimed is:

1. An anchor and interbody for interbody fusion, comprising:
an interbody for insertion between vertebrae of a human spine, comprising an interbody cage comprising a plurality of anchor receptacles, each anchor receptacle configured to receive an anchor to secure the interbody to a vertebra; and
the anchor, comprising:
a head configured to engage one of the plurality of anchor receptacles;
a curved partial-tubular shank with a convex curve, connected to or integral with the head at a distal end of the head, and extending from the head;
a tapered tip portion extending from a distal end of the curved partial-tubular shank;
one or more rails integral with or connected to an outside surface of the anchor, extending from a proximal end of head and along the curved partial-tubular shank to a proximal end of the tapered tip portion; and
a feature formed from two flat surfaces on an outside of the anchor and a rail at an apex where the two surfaces meet.

2. The anchor and interbody of claim 1, wherein each of the rails is a cornered rail or a smooth rail.

3. The anchor and interbody of claim 1, wherein, when there are two or more rails, the rails can be separated around the outside surface by an angle of up to 180°.

4. The anchor and interbody of claim 1, wherein the anchor further comprises a plurality of teeth disposed on an edge of the curved partial-tubular shank.

5. The anchor and interbody of claim 1, wherein the head comprises a threaded opening.

6. The anchor and interbody of claim 1, wherein the interbody, the anchor, or both can be made of biocompatible materials, such as, stainless steel, titanium alloys, aluminum alloys, chromium alloys, metal alloys, CoCrMo, hydroxyapetite, polyether ether ketone (PEEK), polyether ketone ketone (PEKK), carbon fiber, ABS plastic, polyurethane, polyethylene, photo-polymer, resin, fiber-encased resinous material, a polymer, natural materials, other biocompatible materials, and combinations thereof.

7. The anchor and interbody of claim 1, wherein the anchor, the interbody, or both can be produced at least in part by traditional subtractive manufacturing, rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.

8. An anchor and interbody kit comprising:
an interbody for insertion between vertebrae of a human spine, comprising an interbody cage comprising a plurality of anchor receptacles, each anchor receptacle configured to receive an anchor to secure the interbody to a vertebra;
the anchor, comprising:
a head configured to engage one of the plurality of anchor receptacles;
a curved partial-tubular shank with a convex curve, connected to or integral with the head at a distal end of the head, and extending from the head;
a tapered tip portion extending from a distal end of the curved partial-tubular shank;
one or more rails integral with or connected to an outside surface of the anchor, extending from a proximal end of head and along the curved partial-tubular shank to a proximal end of the tapered tip portion; and
a feature formed from two flat surfaces on the outside of the anchor and a rail at an apex where the two surfaces meet; and
one or more tools for manipulating the anchor, the interbody, or both.

9. The anchor and interbody kit of claim 8, wherein each of the rails is a cornered rail or a smooth rail.

10. The anchor and interbody kit of claim 8, wherein, when there are two or more rails, the rails can be separated around the outside surface by an angle of up to 180°.

11. The anchor and interbody kit of claim 8, wherein the anchor further comprises a plurality of teeth disposed on an edge of the curved partial-tubular shank.

12. The anchor and interbody kit of claim 8, wherein the head comprises a threaded opening.

13. The anchor and interbody kit of claim 8, wherein the interbody, the anchor, or both can be made of biocompatible materials, such as, stainless steel, titanium alloys, aluminum alloys, chromium alloys, metal alloys, CoCrMo, hydroxyapetite, polyether ether ketone (PEEK), polyether ketone ketone (PEKK), carbon fiber, ABS plastic, polyurethane, polyethylene, photo-polymer, resin, fiber-encased resinous material, a polymer, natural materials, other biocompatible materials, and combinations thereof.

14. The anchor and interbody kit of claim 8, wherein the anchor, the interbody, or both can be produced at least in part by traditional subtractive manufacturing, rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.

15. A method for using an anchor and interbody for interbody fusion, comprising:
providing a patient in need of an interbody fusion;
providing an interbody for insertion between vertebrae of a human spine, comprising an interbody cage comprising a plurality of anchor receptacles, each anchor receptacle configured to receive an anchor to secure the interbody to a vertebra;
providing the anchor, comprising:
a head configured to engage one of the plurality of anchor receptacles;
a curved partial-tubular shank with a convex curve, connected to or integral with the head at a distal end of the head, and extending from the head;
a tapered tip portion extending from a distal end of the curved partial-tubular shank;
one or more rails integral with or connected to an outside surface of the anchor, extending from a proximal end of head and along the curved partial-tubular shank to a proximal end of the tapered tip portion; and
a feature formed from two flat surfaces on an outside of the anchor and a rail at an apex where the two surfaces meet;
inserting the interbody between two vertebrae of the patient; and
inserting the anchor into one of the plurality of anchor receptacles to secure the interbody to a vertebra.

16. The method of claim 15, wherein each of the rails is a cornered rail or a smooth rail.

17. The method of claim 15, wherein, when there are two or more rails, the rails can be separated around the outside surface by an angle of up to 180°.

18. The method of claim 15, wherein the anchor further comprises a plurality of teeth disposed on an edge of the curved partial-tubular shank.

19. The method of claim 15, wherein the head comprises a threaded opening.

20. The method of claim 15, wherein the interbody, the anchor, or both can be made of biocompatible materials, such as, stainless steel, titanium alloys, aluminum alloys, chromium alloys, metal alloys, CoCrMo, hydroxyapetite, polyether ether ketone (PEEK), polyether ketone (PEKK), carbon fiber, ABS plastic, polyurethane, polyethylene, photo-polymer, resin, fiber-encased resinous material, a polymer, natural materials, other biocompatible materials, and combinations thereof.

21. The method of claim 15, wherein the anchor, the interbody, or both can be produced at least in part by traditional subtractive manufacturing, rapid prototyping, 3D printing, stereolithography (STL), selective laser sintering (SLS), fused deposition modeling (FDM), direct metal laser sintering (DMLS), electron beam melting (EBM), multi-jet fusion (MJF), or an additive manufacturing machine.
